(19) United States
(12) Patent Application Publication
(10) Pub. No.: US 2016/0280807 A1
(43) Pub. Date: Sep. 29, 2016

(54) PREPARATION OF NON-ISOCYANATE URETHANE (METH) ACRYLATES FOR URETHANE FUNCTIONAL LATEX

(71) Applicants: Mark D. Soucek, Cuyahoga Falls, OH (US); Lei Meng, Akron, OH (US)

(72) Inventors: Mark D. Soucek, Cuyahoga Falls, OH (US); Lei Meng, Akron, OH (US)

(21) Appl. No.: 15/079,818

(22) Filed: Mar. 24, 2016

Related U.S. Application Data
(60) Provisional application No. 62/137,303, filed on Mar. 24, 2015.

(51) Int. Cl. C08F 2/22; C07C 271/12

(52) U.S. Cl. CPC C08F 2/22; C07C 271/12

(57) ABSTRACT

A urethane-functional (meth)acrylate monomer is provided that is defined by the formula

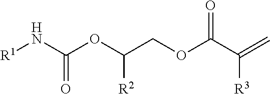

where R¹ is a monovalent organic group, R² is a hydrogen atom or a monovalent organic group, and R³ is a hydrogen atom or an alkyl group. The urethane-functional (meth)acrylate monomer may be polymerized and advantageously provides improved mechanical properties such as tensile modulus, tensile strength and elongation-at-break tensile modulus, tensile strength and elongation-at-break.

### CROSS-REFERENCE TO RELATED APPLICATIONS

The present application claims priority to U.S. Provisional Patent Application No. 62/137,303, filed Mar. 24, 2015, which is incorporated herein by reference.

### FIELD OF THE INVENTION

Embodiments are directed to urethane-functional (meth)acrylate monomers, the polymerization of urethane-functional (meth)acrylate monomers, and the resultant polymers. When a urethane-functional (meth)acrylate monomer is polymerized using emulsion polymerization techniques, a latex may be prepared that includes a unit derived from a urethane-functional (meth)acrylate monomer.

### BACKGROUND OF THE INVENTION

Environmentally friendly coatings with low volatile organic compounds have attracted lots of attention in the past decades. Waterborne coatings have been irreplaceable due to the advantages of the use of water as the solvent. Polyacrylates and polyurethanes are two of the most popular resins that are widely used in waterborne coating systems. Polyacrylates and polyurethanes both have their own characteristic properties related with their chemical structures. The main polymer chain of a polyacrylate consists of carbon-carbon bonds, leading to advantages such as excellent water and chemical resistance, weathering properties, and hardness. In addition, polyacrylates are cost friendly. Polyacrylates have been widely used for coatings, paper and textile finishes, adhesives, and other applications since their introduction in the 1950s. However, polyacrylates have some disadvantages, which limit their usage in some specific applications, such as those that require high elasticity and abrasion resistance. Polyurethanes, which have been used in various coatings since 1970s, possess excellent elasticity, scratch resistance, flexibility and toughness. This is the result of the morphology of hard/soft domains along with the acyclic and/or cyclic intermolecular hydrogen bonds between polymer molecules. However, the hardness and the water and alkali resistance of polyurethanes are inferior to that of polyacrylates. Moreover, the cost of polyurethanes, especially the aliphatic isocyanate based polyurethanes, is much higher than that of polyacrylates.

Combining polyurethanes and polyacrylates has been proposed in an attempt combine the beneficial properties of each polymer for specific applications. Although some benefits have been obtained from this approach, properties of the resulting physical blends do not match up to expected values as predicted from the simple “rule of mixtures.” In many cases, these blends compromise the superior performance properties of one or both polymers. The reasons for these types of undesired effects with blends have not been well defined. One possible reason for this behavior is that, on a molecular level, the acrylic polymers are not soluble in the polyurethane polymers. Therefore, the acrylic/polyurethane polymers remain phase separated during film formation, in which the different polymers are present in separate particles. This may explain why direct blending of acrylic emulsions and polyurethanes results in poor properties.

A more widely used method for combining polyurethane and polyacrylate is to synthesize a hybrid polyurethane/polyacrylate. In past decades, various methods of preparing a hybrid polyurethane/polyacrylate have been explored, including intimately mixing, grafting, interpenetrating polymer networks and producing particles with a core-shell morphology. The most representative approach is to polymerize acrylic monomers in the presence of a polyurethane dispersion to obtain hybrid emulsions. Generally, these process require the use of isocyantes, which are toxic. The human body contains proteins and other materials having the substituents, such as hydroxyl, amine, and/or carboxylic acid groups that can be reacted with the isocyanates. The toxicity of isocyantes requires that they be stored, handled and processed with secure industrial equipment, following special safety procedures. Which increase the cost of the polyurethane.

Presently, there is a need in the art for a polymer that combines the polymer properties of polyacrylates and polyurethanes and that may be prepared without the use of isocyanate compounds.

### SUMMARY OF THE INVENTION

In a first embodiment, the present provides a monomer defined by the formula

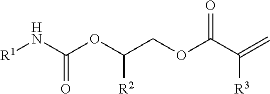

where R¹ is a monovalent organic group, R² is a hydrogen atom or a monovalent organic group, and R³ is a hydrogen atom or an alkyl group. In these or other embodiments, the alkyl group, R³, is a methyl group.

In a second embodiment, the present provides a monomer as in the first embodiment, where the organic group, R¹, is a hydrocarbon group with 1 carbon atom to about 12 carbon atoms.

In a third embodiment, the present provides a monomer as in the first or second embodiments, where the organic group, R², is a hydrocarbon group with 1 carbon atom to about 12 carbon atoms.

In a fourth embodiment, the present provides a monomer as in any of the first through third embodiments, where R¹ is a hydrocarbon group selected from methyl, ethyl, propyl, isopropyl, isobutyl, tert-butyl, n-butyl, sec-butyl, isopentyl, tertpentyl, n-pentyl, sec-pentyl, terthexyl, n-hexyl, isohexyl, and sec-hexyl, n-heptyl, n-octyl, n-nonyl, n-decyl and n-dodecyl.

In a fifth embodiment, the present provides a monomer as in any of the first through fourth embodiments, where R³ is a hydrogen atom.

In a sixth embodiment, the present provides a monomer as in any of the first through fifth embodiments, where R³ is a methyl group.

In a seventh embodiment, the present provides a monomer as in any of the first through sixth embodiments, where the monomer is selected from:

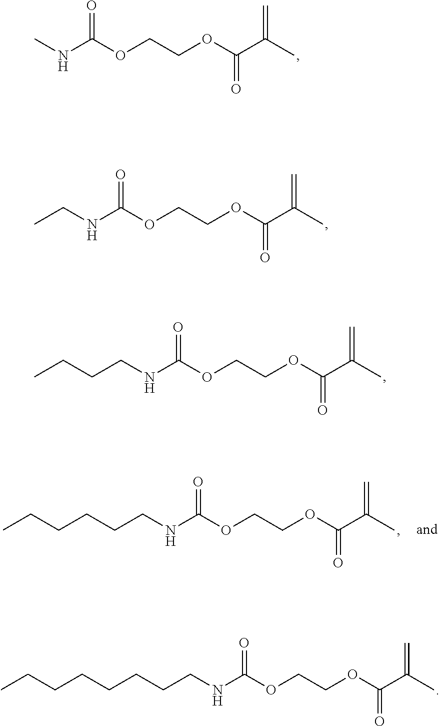

In an eighth embodiment, provides a method of preparing a polymer comprising:

polymerizing a urethane-functional acrylate monomer defined by the formula

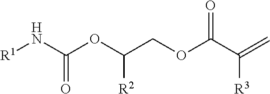

where R¹ is a monovalent organic group, R² is a hydrogen atom or a monovalent organic group, and R³ is a hydrogen atom or an alkyl group. In certain embodiments, the alkyl group, R³, is a methyl group.

In a ninth embodiment, the present provides a method as in the eighth embodiment, where the polymerization is initiated with a radical initiator.

In a tenth embodiment, the present provides a method as in either the eighth or ninth embodiments, where the polymerization is a controlled living polymerization.

In an eleventh embodiment, the present provides a method as in any of the eighth through tenth embodiments, where the controlled living polymerization is selected from atom transfer radical polymerization, reverse atom transfer radical polymerization, reversible addition-fragmentation chain-transfer polymerization, and nitroxide mediated polymerization.

In a twelfth embodiment, the present provides a method as in any of the eighth through eleventh embodiments, where the polymerization is performed as an emulsion polymerization.

In a thirteenth embodiment, the present provides a method as in any of the eighth through twelfth embodiments, where the emulsion polymerization produces a core-shell polymer particle.

In a fourteenth embodiment, the present provides a method as in any of the eighth through thirteenth embodiments, where the polymerization includes one or more co-monomers.

In a fifteenth embodiment, the present provides a method as in any of the eighth through fourteenth embodiments, where the co-monomers are selected from (meth)acrylic acids, (meth)acrylates, and vinyl aromatic compounds.

In a sixteenth embodiment, a polymer is provided with a unit defined by the formula:

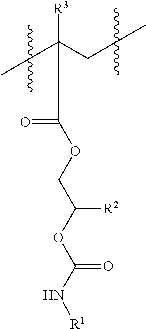

where R¹ is a monovalent organic group, R² is a hydrogen atom or a monovalent organic group, and R³ is a hydrogen atom or an alkyl group.

In a seventeenth embodiment, a polymer as in sixteenth embodiment, where the polymer is defined by the formula

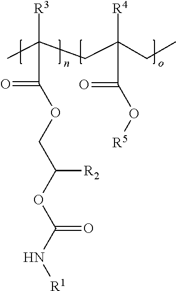

where each R¹ is individually a monovalent organic group, each R² is a individually hydrogen atom or a monovalent organic group, each R³ is individually a hydrogen atom or an alkyl group, each R⁴ is individually a hydrogen atom or an alkyl group, each R⁵ is individually a hydrogen atom or a monovalent organic group, n is from about 5 units to about 500 units, and o is from about 50 units to about 5000 units. In these or other embodiments, the alkyl group, R³ or R⁴, is a methyl group.

In an eighteenth embodiment, a polymer is provided as in the sixteenth or seventeenth embodiments, where the polymer is defined by the formula

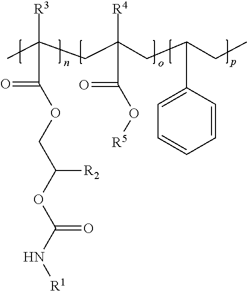

where each R¹ is individually a monovalent organic group, each R² is a individually hydrogen atom or a monovalent organic group, each R³ is individually a hydrogen atom or an alkyl group, each R⁴ is individually a hydrogen atom or an alkyl group, each R⁵ is individually a hydrogen atom or an organic group, n is from about 5 units to about 500 units, o is from about 50 units to about 5000 units, and p is from about 50 units to about 5000 units.

In a nineteenth embodiment, a polymer is provided as in any of the sixteenth through eighteenth embodiments, where the polymer is defined by the formula

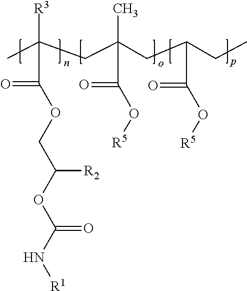

where each R¹ is individually a monovalent organic group, each R² is a individually hydrogen atom or a monovalent organic group, each R³ is individually a hydrogen atom or an alkyl group, each R⁵ is individually a hydrogen atom or a monovalent organic group, n is from about 5 units to about 500 units, o is from about 50 units to about 5000 units, and p is from about 50 units to about 5000 units. In a twentieth embodiment, a latex is provided comprising a polymer particle with a unit defined by the formula:

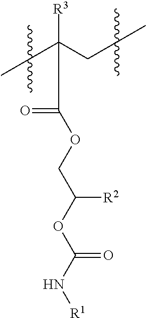

where R¹ is an organic group, where R² is a hydrogen atom or an organic group, and R³ is a hydrogen atom or an alkyl group. In these or other embodiments, the alkyl group, R³, is a methyl group.

### BRIEF DESCRIPTION OF THE DRAWINGS

FIG. 1 provides a schematic for the synthesis of a urethane-functional (meth)acrylate monomer.

FIG. 2 provides a chart of the stress-strain curves of BFL and SBFL10% latex films

FIG. 3A provides a chart of the storage modulus curves for BFL and SBFL10% latex films.

FIG. 3B provides a chart of the tan δ curves for BFL and SBFL10% latex films.

FIG. 4A provides a TEM image of the core-shell morphology for sample of a FLC-S20% latex. The black bar represents 500 nm.

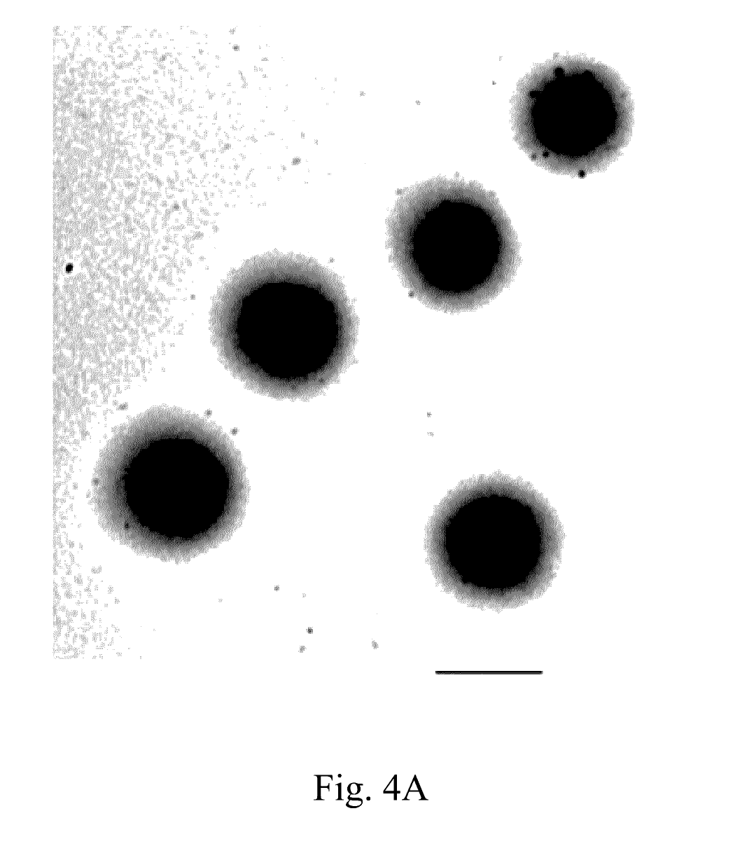

FIG. 4B provides a TEM image of the core-shell morphology for sample of a C-FLS20% latex. The black bar represents 500 nm.

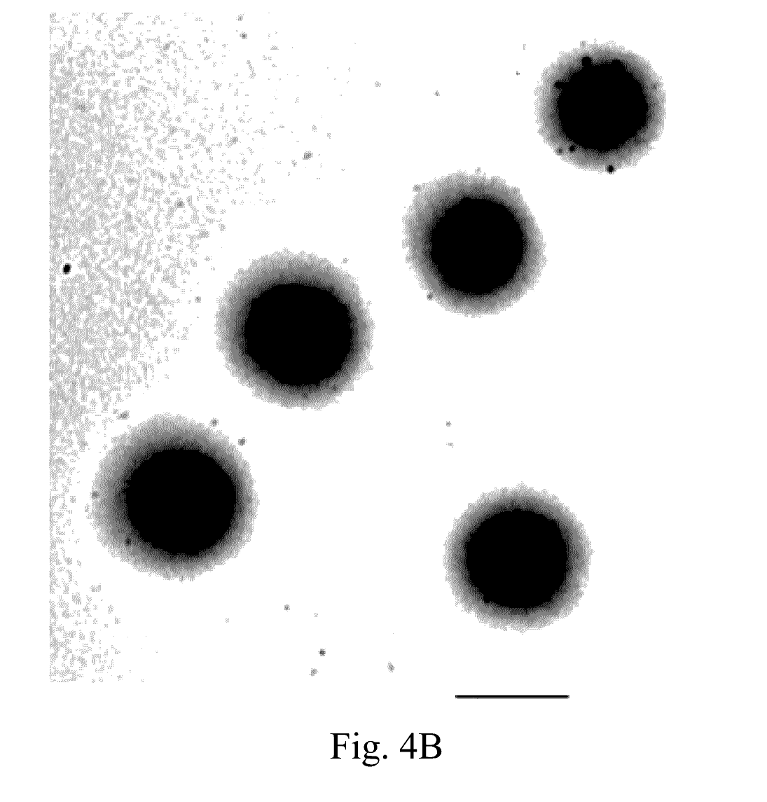

FIG. 4C provides a TEM image of the core-shell morphology for sample of a FLH10% latex. The black bar represents 500 nm.

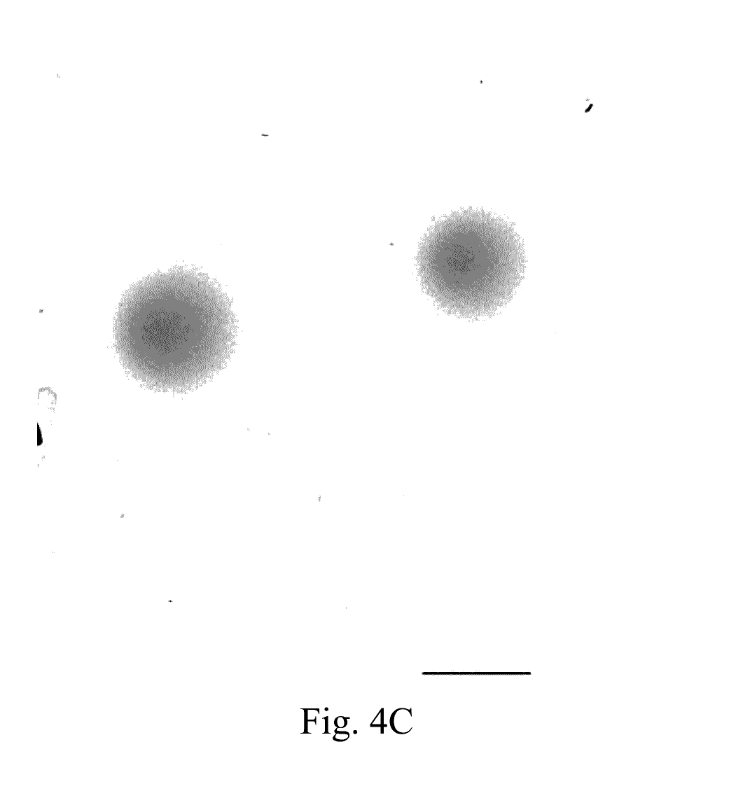

FIG. 5A provides a graph of the modulus for core-shell and homogeneous latexes.

FIG. 5B provides a graph of the tensile strength for core-shell and homogeneous latexes.

FIG. 5C provides a graph of the elongation-at-break for core-shell and homogeneous latexes.

### DETAILED DESCRIPTION OF ILLUSTRATIVE EMBODIMENTS

One or more embodiments are based upon the discovery that an (meth)acrylate monomer that includes a urethane group (carbamate group) may be prepared without the use of an isocyanate compound. Advantageously, polymers prepared with a non-isocyanate urethane (meth)acrylates produce poly(meth)acrylates with an increased Tg when compared with a similar polyacrylate prepared with an acrylate such as a methyl methcrylate. The non-isocyanate urethane poly(meth)acrylates also have improved mechanical properties such as tensile modulus, tensile strength and elongation-at-break when compared to similar polymers prepared with methyl methacrylate. While not wishing to be bound by any particular theory or mechanism, it is believed that the improved mechanical properties are a result of increased physical interaction forces due to hydrogen bonding among the urethane groups. Poly(meth)acrylates prepared from urethane-functional (meth)acrylate monomer may be used for coatings, finishes, adhesives and other applications.

As noted above, the non-isocyanate urethane (meth)acrylate, which may be referred to a urethane-functional (meth)acrylate monomer, is a (meth)acrylate compound that includes a urethane group. For the purposes of this disclosure the use of the modifier “(meth)” in conjunction with the term “acrylate” is used to include indicate the at acrylate may be an acrylate, methacrylate or a mixture of both the acrylate and methacrylate. In one or more embodiments, the urethane group of the urethane-functional (meth)acrylate monomer is part of an organic group attached to the oxygen atom of the acrylate compound. In one or more embodiments, the urethane-functional (meth)acrylate monomer is defined by the following formula:

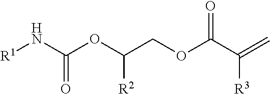

where R¹ is a monovalent organic group, R² is a hydrogen atom or a monovalent organic group, and R³ is a hydrogen atom or an alkyl group. In certain embodiments, the alkyl group, R³, is a methyl group.

Suitable monovalent organic groups for use in a urethane-functional (meth)acrylate monomer include linear, branched or cyclic hydrocarbon groups. The organic group may be a saturated or an unsaturated hydrocarbon group. In one or more embodiments, the monovalent organic groups may be characterized by the number of carbon atoms in the group. In these or other embodiments, the monovalent organic group may include from about 1 to about 12 carbon atoms, in other embodiments from about 3 to about 10 carbon atoms, and in other embodiments from about 5 to about 8 carbon atoms.

Exemplary monovalent organic groups include, but are not limited to, methyl, ethyl, propyl, isopropyl, isobutyl, tert-butyl, n-butyl, sec-butyl, isopentyl, tertpentyl, n-pentyl, sec-pentyl, tert-hexyl, n-hexyl, iso-hexyl, and sec-hexyl, n-heptyl, n-octyl, n-nonyl, n-decyl and n-dodecyl.

Exemplary urethane-functional acrylate monomer include but are not limited to, 2-[(methylcarbamoyl)oxy]ethyl acrylate, 2-[(methylcarbamoyl)oxy]-2-methylethyl acrylate, 2-[(methylcarbamoyl)oxy]-2-ethylethyl acrylate, 2-[(methylcarbamoyl)oxy]-2-isopropylethyl acrylate, 2-[(ethylcarbamoyl)oxy]ethyl acrylate, 2-[(ethylcarbamoyl)oxy]-2-methylethyl acrylate, 2-[(ethylcarbamoyl)oxy]-2-ethylethyl acrylate, 2-[ethylcarbamoyl)oxy]-2-isopropylethyl acrylate, 2-[(propylcarbamoyl)oxy]ethyl acrylate, 2-[(propylcarbamoyl)oxy]-2-methylethyl acrylate, 2-[(propylcarbamoyl)oxy]-2-ethylethyl acrylate, 2-[(propylcarbamoyl)oxy]-2-isopropylethyl acrylate, 2-[(isopropylcarbamoyl)oxy]ethyl acrylate, 2-[(isopropylcarbamoyl)oxy]-2-methylethyl acrylate, 2-[(isopropylcarbamoyl)oxy]-2-ethylethyl acrylate, 2-[(isopropylcarbamoyl)oxy]-2-isopropylethyl acrylate, 2-[(isobutylcarbamoyl)oxy]ethyl acrylate, 2-[(isobutylcarbamoyl)oxy]-2-methylethyl acrylate, 2-[(isobutylcarbamoyl)oxy]-2-ethylethyl acrylate, 2-[(isobutylcarbamoyl)oxy]-2-isopropylethyl acrylate, 2-[(tert-butylcarbamoyl)oxy]ethyl acrylate, 2-[(tert-butylcarbamoyl)oxy]-2-methylethyl acrylate, 2-[(tert-butylcarbamoyl)oxy]-2-ethylethyl acrylate, 2-[(tert-butylcarbamoyl)oxy]-2-isopropylethyl acrylate, 2-[(sec-butylcarbamoyl)oxy]ethyl acrylate, 2-[(sec-butylcarbamoyl)oxy]-2-methylethyl acrylate, 2-[(sec-butylcarbamoyl)oxy]-2-ethylethyl acrylate, 2-[(sec-butylcarbamoyl)oxy]-2-isopropylethyl acrylate, 2-[(n-butylcarbamoyl)oxy]ethyl acrylate, 2-[(n-butylcarbamoyl)oxy]-2-methylethyl acrylate, 2-[(n-butylcarbamoyl)oxy]-2-ethylethyl acrylate, 2-[(n-butylcarbamoyl)oxy]-2-isopropylethyl acrylate, 2-[(n-pentylcarbamoyl)oxy]ethyl acrylate, 2-[(n-pentylcarbamoyl)oxy]-2-methylethyl acrylate, 2-[(n-pentylcarbamoyl)oxy]-2-ethylethyl acrylate, 2-[(n-pentylcarbamoyl)oxy]-2-isopropylethyl acrylate, 2-[(sec-pentylcarbamoyl)oxy]ethyl acrylate, 2-[(sec-pentylcarbamoyl)oxy]-2-methylethyl acrylate, 2-[(sec-pentylcarbamoyl)oxy]-2-ethylethyl acrylate, 2-[(sec-pentylcarbamoyl)oxy]-2-isopropylethyl acrylate, 2-[(tert-hexylcarbamoyl)oxy]ethyl acrylate, 2-[(tert-hexylcarbamoyl)oxy]-2-methylethyl acrylate, 2-[(tert-hexylcarbamoyl)oxy]-2-ethylethyl acrylate, 2-[(tert-hexylcarbamoyl)oxy]-2-isopropylethyl acrylate, 2-[(n-hexylcarbamoyl)oxy]ethyl acrylate, 2-[(n-hexylcarbamoyl)oxy]-2-methylethyl acrylate, 2-[(n-hexylcarbamoyl)oxy]-2-ethylethyl acrylate, 2-[(n-hexylcarbamoyl)oxy]-2-isopropylethyl acrylate, 2-[(iso-hexylcarbamoyl)oxy]ethyl acrylate, 2-[(iso-hexylcarbamoyl)oxy]-2-methylethyl acrylate, 2-[(iso-hexylcarbamoyl)oxy]-2-ethylethyl acrylate, 2-[(iso-hexylcarbamoyl)oxy]-2-isopropylethyl acrylate, 2-[(sec-hexylcarbamoyl)oxy]ethyl acrylate, 2-[(sec-hexylcarbamoyl)oxy]-2-methylethyl acrylate, 2-[(sec-hexylcarbamoyl)oxy]-2-ethylethyl acrylate, 2-[(sec-hexylcarbamoyl)oxy]-2-isopropylethyl acrylate, 2-[(n-heptylcarbamoyl)oxy]ethyl acrylate, 2-[(n-heptylcarbamoyl)oxy]-2-methylethyl acrylate, 2-[(n-heptylcarbamoyl)oxy]-2-ethylethyl acrylate, 2-[(n-heptylcarbamoyl)oxy]-2-isopropylethyl acrylate, 2-[(n-octylcarbamoyl)oxy]ethyl acrylate, 2-[(n-octylcarbamoyl)oxy]-2-methylethyl acrylate, 2-[(n-octylcarbamoyl)oxy]-2-ethylethyl acrylate, 2-[(n-octylcarbamoyl)oxy]-2-isopropylethyl acrylate, 2-[(n-nonylcarbamoyl)oxy]ethyl acrylate, 2-[(n-nonylcarbamoyl)oxy]-2-methylethyl acrylate, 2-[(n-nonylcarbamoyl)oxy]-2-ethylethyl acrylate, 2-[(n-nonylcarbamoyl)oxy]-2-isopropylethyl acrylate, 2-[(n-decylcarbamoyl)oxy]ethyl acrylate, 2-[(n-decylcarbamoyl)oxy]-2-methylethyl acrylate, 2-[(n-decylcarbamoyl)oxy]-2-ethylethyl acrylate, 2-[(n-decylcarbamoyl)oxy]-2-isopropylethyl acrylate, 2-[(n-dodecylcarbamoyl)oxy]ethyl acrylate, 2-[(n-dodecylcarbamoyl)oxy]-2-methylethyl acrylate, 2-[(n-dodecylcarbamoyl)oxy]-2-ethylethyl acrylate, and 2-[(n-dodecylcarbamoyl)oxy]-2-isopropylethyl acrylate.

Exemplary urethane-functional methacrylate monomer include but are not limited to, 2-[(methylcarbamoyl)oxy]ethyl methacrylate, 2-[(methylcarbamoyl)oxy]-2-methylethyl methacrylate, 2-[(methylcarbamoyl)oxy]-2-ethylethyl methacrylate, 2-[(methylcarbamoyl)oxy]-2-isopropylethyl methacrylate, 2-[(ethylcarbamoyl)oxy]ethyl methacrylate, 2-[(ethylcarbamoyl)oxy]-2-methylethyl methacrylate, 2-[(ethylcarbamoyl)oxy]-2-ethylethyl methacrylate, 2-[(ethylcarbamoyl)oxy]-2-isopropylethyl methacrylate, 2-[(propylcarbamoyl)oxy]ethyl methacrylate, 2-[(propylcarbamoyl)oxy]-2-methylethyl methacrylate, 2-[(propylcarbamoyl)oxy]-2-ethylethyl methacrylate, 2-[(propylcarbamoyl)oxy]-2-isopropylethyl methacrylate, 2-[(isopropylcarbamoyl)oxy]ethyl methacrylate, 2-[(isopropylcarbamoyl)oxy]-2-methylethyl methacrylate, 2-[(isopropylcarbamoyl)oxy]-2-ethylethyl methacrylate, 2-[(isopropylcarbamoyl)oxy]-2-isopropylethyl methacrylate, 2-[(isobutylcarbamoyl)oxy]ethyl methacrylate, 2-[(isobutylcarbamoyl)oxy]-2-methylethyl methacrylate, 2-[(isobutylcarbamoyl)oxy]-2-ethylethyl methacrylate, 2-[(isobutylcarbamoyl)oxy]-2-isopropylethyl methacrylate, 2-[tert-butylcarbamoyl)oxy]ethyl methacrylate, 2-[(tert-butylcarbamoyl)oxy]-2-methylethyl methacrylate, 2-[(tert-butylcarbamoyl)oxy]-2-ethylethyl methacrylate, 2-[(tert-butylcarbamoyl)oxy]-2-isopropylethyl methacrylate, 2-[(sec-butylcarbamoyl)oxy]ethyl methacrylate, 2-[(sec-butylcarbamoyl)oxy]-2-methylethyl methacrylate, 2-[(sec-butylcarbamoyl)oxy]-2-ethylethyl methacrylate, 2-[(sec-butylcarbamoyl)oxy]-2-isopropylethyl methacrylate, 2-[(n-butylcarbamoyl)oxy]ethyl methacrylate, 2-[(n-butylcarbamoyl)oxy]-2-methylethyl methacrylate, 2-[(n-butylcarbamoyl)oxy]-2-ethylethyl methacrylate, 2-[(n-butylcarbamoyl)oxy]-2-isopropylethyl methacrylate, 2-[(n-pentylcarbamoyl)oxy]ethyl methacrylate, 2-[(n-pentylcarbamoyl)oxy]-2-methylethyl methacrylate, 2-[(n-pentylcarbamoyl)oxy]-2-ethylethyl methacrylate, 2-[(n-pentylcarbamoyl)oxy]-2-isopropylethyl methacrylate, 2-[(sec-pentylcarbamoyl)oxy]ethyl methacrylate, 2-[(sec-pentylcarbamoyl)oxy]-2-methylethyl methacrylate, 2-[(sec-pentylcarbamoyl)oxy]-2-ethylethyl methacrylate, 2-[(sec-pentylcarbamoyl)oxy]-2-isopropylethyl methacrylate, 2-[(tert-hexylcarbamoyl)oxy]ethyl methacrylate, 2-[(tert-hexylcarbamoyl)oxy]-2-methylethyl methacrylate, 2-[(tert-hexylcarbamoyl)oxy]-2-ethylethyl methacrylate, 2-[(tert-hexylcarbamoyl)oxy]-2-isopropylethyl methacrylate, 2-[(n-hexylcarbamoyl)oxy]ethyl methacrylate, 2-[(n-hexylcarbamoyl)oxy]-2-methylethyl methacrylate, 2-[(n-hexylcarbamoyl)oxy]-2-ethylethyl methacrylate, 2-[(n-hexylcarbamoyl)oxy]-2-isopropylethyl methacrylate, 2-[(iso-hexylcarbamoyl)oxy]ethyl methacrylate, 2-[(iso-hexylcarbamoyl)oxy]-2-methylethyl methacrylate, 2-[(iso-hexylcarbamoyl)oxy]-2-ethylethyl methacrylate, 2-[(iso-hexylcarbamoyl)oxy]-2-isopropylethyl methacrylate, 2-[(sec-hexylcarbamoyl)oxy]ethyl methacrylate, 2-[(sec-hexylcarbamoyl)oxy]-2-methylethyl methacrylate, 2-[(sec-hexylcarbamoyl)oxy]-2-ethylethyl methacrylate, 2-[(sec-hexylcarbamoyl)oxy]-2-isopropylethyl methacrylate, 2-[(n-heptylcarbamoyl)oxy]ethyl methacrylate, 2-[(n-heptylcarbamoyl)oxy]-2-methylethyl methacrylate, 2-[(n-heptylcarbamoyl)oxy]-2-ethylethyl methacrylate, 2-[(n-heptylcarbamoyl)oxy]-2-isopropylethyl methacrylate, 2-[(n-octylcarbamoyl)oxy]ethyl methacrylate, 2-[(n-octylcarbamoyl)oxy]-2-methylethyl methacrylate, 2-[(n-octylcarbamoyl)oxy]-2-ethylethyl methacrylate, 2-[(n-octylcarbamoyl)oxy]-2-isopropylethyl methacrylate, 2-[n-nonylcarbamoyl)oxy]ethyl methacrylate, 2-[(n-nonylcarbamoyl)oxy]-2-methylethyl methacrylate, 2-[(n-nonylcarbamoyl)oxy]-2-ethylethyl methacrylate, 2-[(n-nonylcarbamoyl)oxy]-2-isopropylethyl methacrylate, 2-[(n-decylcarbamoyl)oxy]ethyl methacrylate, 2-[(n-decylcarbamoyl)oxy]-2-methylethyl methacrylate, 2-[(n-decylcarbamoyl)oxy]-2-ethylethyl methacrylate, 2-[(n-decylcarbamoyl)oxy]-2-isopropylethyl methacrylate, 2-[(n-dodecylcarbamoyl)oxy]ethyl methacrylate, 2-[(n-dodecylcarbamoyl)oxy]-2-methylethyl methacrylate, 2-[(n-dodecylcarbamoyl)oxy]-2-ethylethyl methacrylate, and 2-[(n-dodecylcarbamoyl)oxy]-2-isopropylethyl methacrylate.

In one or more embodiments, the urethane-functional (meth)acrylate monomer may be defined by one or more of the following structures:

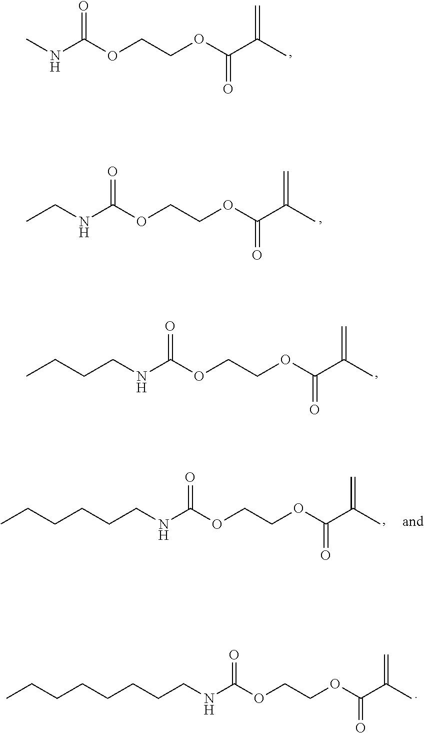

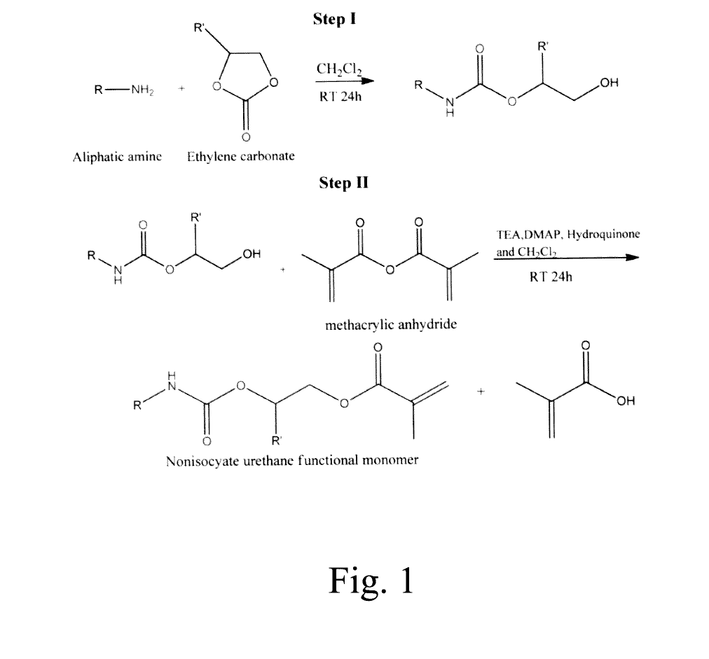

A general reaction scheme for preparing a urethane-functional (meth)acrylate monomer is provided in FIG. 1, and further specifics are provided in the experimental section herein. In one or more embodiments, the urethane-functional (meth)acrylate monomer may be prepared in a two-step process. In these or other embodiments, the urethane forming reaction involves an attack by a nucleophile (the amine group) at the electrophilic carbon of a carbonate compound. Suitable carbonate compounds include ethylene carbonates, which are particularly advantageous because there no substituent effects when they are used. In a second reaction, the compound with an alcohol group and a carbonate group is reacted with an (meth)acrylate or (meth)acrylic anhydride via a acetylation of the alcohol adduct to produce the urethane-functional (meth)acrylate monomer. In one or more embodiments, the second step may be catalyzed by DMAP. In these or other embodiments, the urethane-functional (meth)acrylate monomer is prepared without the use of an isocyanate compound.

Advantageously, a urethane-functional (meth)acrylate monomer may be used in any conventional (meth)acrylate polymerization system. In these or other embodiments, a urethane-functional (meth)acrylate monomer may be substituted for all or part of a (meth)acrylate monomer in a typical process for preparing a poly(meth)acrylate polymer or copolymer. As those skilled in the art will appreciate, the urethane-functional (meth)acrylate monomer, and optionally any co-monomers, may be polymerized by different polymerization systems. In one or more embodiments, the urethane-functional (meth)acrylate monomer (and optionally co-monomer) may be polymerized in a radical polymerization. In one or more embodiments, the polymerization system is a controlled living polymerization. Examples of controlled living polymerization systems include atom transfer radical polymerization, reverse atom transfer radical polymerization, reversible addition-fragmentation chain-transfer polymerization, and nitroxide mediated polymerization. In these or other embodiments, the polymerization of the urethane-functional (meth)acrylate monomer (and optionally co-monomer) may be performed as an emulsion polymerization.

The urethane-functional (meth)acrylate monomer may be polymerized along with co-monomers. Suitable co-monomers include compounds that have a carbon-carbon double bond that may react with an acrylate group. Examples of co-monomers include, but are not limited to, (meth)acrylic acids, (meth)acrylates, and vinyl aromatic compounds.

In one or more embodiments, the urethane-functional (meth)acrylate monomer may be polymerized along with an acrylic acid co-monomer. In one or more embodiments, the urethane-functional (meth)acrylate monomer may be polymerized along with a methacrylic acid co-monomer. In one or more embodiments, the urethane-functional (meth)acrylate monomer may be polymerized along with one or more acrylate co-monomers. In one or more embodiments, the urethane-functional (meth)acrylate monomer may be polymerized along with one or more methacrylate co-monomers. In one or more embodiments, the urethane-functional (meth)acrylate monomer may be polymerized along with one or more vinyl aromatic compounds. In one or more embodiments, the urethane-functional (meth)acrylate monomer may be polymerized along with monomer selected from the group consisting of acrylic acid, methacrylic acid, one or more acrylate co-monomers, one or more methacrylate co-monomers, one or more vinyl aromatic compounds, or any combination thereof.

The term (meth)acrylate has is used indicate conventional acrylate monomers. These may include acrylates with organic or crosslinkable groups. In one or more embodiments, the (meth)acrylate may be used to describe organic (meth)acrylate, which include alkyl acrylates.

Exemplary acrylate compounds include, but are not limited to, methyl acrylate, ethyl acrylate, propyl acrylate, isopropyl acrylate, isobutyl acrylate, tert-butyl acrylate, sec-butyl acrylate, n-butyl acrylate, n-pentyl acrylate, sec-pentyl acrylate, tert-hexyl acrylate, n-hexyl acrylate, iso-hexyl acrylate, sec-hexyl acrylate, n-heptyl acrylate, n-octyl acrylate, n-nonyl acrylate, n-decyl acrylate, and n-dodecyl acrylate.

Exemplary methacrylate compounds include, but are not limited to, methyl methacrylate, ethyl methacrylate, propyl methacrylate, isopropyl methacrylate, isobutyl methacrylate, tert-butyl methacrylate, sec-butyl methacrylate, n-butyl methacrylate, n-pentyl methacrylate, sec-pentyl methacrylate, tert-hexyl methacrylate, n-hexyl methacrylate, iso-hexyl methacrylate, sec-hexyl methacrylate, n-heptyl methacrylate, n-octyl methacrylate, n-nonyl methacrylate, n-decyl methacrylate, and n-dodecyl methacrylate.

Exemplary vinyl aromatic compounds include, but are not limited to, styrene, tert-butylstyrene, α-methylstyrene, p-methylstyrene, p-ethylstyrene, divinylbenzene, 1,1-diphenylstyrene, vinylnaphthalene, vinylanthracene, N,N-diethyl-p-aminoethylstyrene, vinylpyridine.

In one or more embodiments, when a urethane-functional (meth)acrylate monomer is used to prepare a copolymer the copolymer may be characterized by the parts per hundred by weight of the urethane-functional (meth)acrylate monomer used in the monomer mixture to prepare the copolymer. In one or more embodiments, the copolymer may be from about 0.5 part to about 99 parts, in other embodiments from about 1 parts to about 90 parts, in other embodiments from about 2 parts to about 80 parts, in other embodiments from about 3 parts to about 50 parts, in other embodiments from about 5 parts to about 35 parts, in other embodiments from about 10 parts to about 25 parts, and in other embodiments from about 15 parts to about 20 parts per hundred urethane-functional (meth)acrylate monomer.

In one or more embodiments, the urethane-functional (meth)acrylate monomer may be part of a copolymer along with an acrylic acid. In these or other embodiments, the copolymer may be characterized by the parts per hundred by weight of the acrylic acid used in the monomer mixture to prepare the copolymer. In one or more embodiments, the copolymer may be from about 0.5 part to about 99 parts, in other embodiments from about 3 parts to about 75 parts, in other embodiments from about 5 parts to about 50 parts, and in other embodiments from about 10 parts to about 25 parts per hundred acrylic acid.

In one or more embodiments, the urethane-functional (meth)acrylate monomer may be part of a copolymer along with a methacrylic acid. In these or other embodiments, the copolymer may be characterized by the parts per hundred by weight of the methacrylic acid used in the monomer mixture to prepare the copolymer. In one or more embodiments, the copolymer may be from about 0.5 part to about 99 parts, in other embodiments from about 1 parts to about 90 parts, in other embodiments from about 2 parts to about 80 parts, in other embodiments from about 3 parts to about 50 parts, in other embodiments from about 5 parts to about 35 parts, in other embodiments from about 10 parts to about 25 parts, and in other embodiments from about 15 parts to about 20 parts per hundred methacrylic acid.

In one or more embodiments, the urethane-functional (meth)acrylate monomer may be part of a copolymer along with an acrylate. In these or other embodiments, the copolymer may be characterized by the parts per hundred by weight of the acrylate used in the monomer mixture to prepare the copolymer. In one or more embodiments, the copolymer may be from about 0.5 part to about 99 parts, in other embodiments from about 1 parts to about 90 parts, in other embodiments from about 2 parts to about 80 parts, in other embodiments from about 3 parts to about 50 parts, in other embodiments from about 5 parts to about 35 parts, in other embodiments from about 10 parts to about 25 parts, and in other embodiments from about 15 parts to about 20 parts per hundred acrylate.

In one or more embodiments, the urethane-functional (meth)acrylate monomer may be part of a copolymer along with a methacrylate. In these or other embodiments, the copolymer may be characterized by the parts per hundred by weight of the methacrylate used in the monomer mixture to prepare the copolymer. In one or more embodiments, the copolymer may be from 0.5 part to about 99 parts, in other embodiments from about 1 parts to about 90 parts, in other embodiments from about 2 parts to about 80 parts, in other embodiments from about 3 parts to about 50 parts, in other embodiments from about 5 parts to about 35 parts, in other embodiments from about 10 parts to about 25 parts, and in other embodiments from about 15 parts to about 20 parts per hundred methacrylate.

In one or more embodiments, the urethane-functional (meth)acrylate monomer may be part of a copolymer along with a vinyl aromatic compound. In these or other embodiments, the copolymer may be characterized by the parts per hundred by weight of the vinyl aromatic compound used in the monomer mixture to prepare the copolymer. In one or more embodiments, the copolymer may be from about 0.5 part to about 99 parts, in other embodiments from about 1 parts to about 90 parts, in other embodiments from about 2 parts to about 80 parts, in other embodiments from about 3 parts to about 50 parts, in other embodiments from about 5 parts to about 35 parts, in other embodiments from about 10 parts to about 25 parts, and in other embodiments from about 15 parts to about 20 parts per hundred vinyl aromatic compound.

In one or more embodiments, when a urethane-functional (meth)acrylate monomer is used to prepare a polymer, the polymer may include a unit derived from a urethane-functional (meth)acrylate monomer defined by the formula:

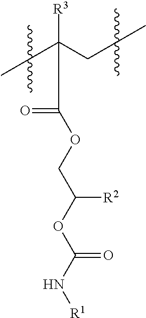

where R¹ is an organic group, R² is a hydrogen atom or an organic group, and R³ is a hydrogen atom or an alkyl group. In certain embodiments, the alkyl group, R³, is a methyl group.

In one or more embodiments, when a urethane-functional (meth)acrylate monomer and a (meth)acrylate or (meth)acrylic acid is used to prepare a polymer, the polymer may be defined by the formula:

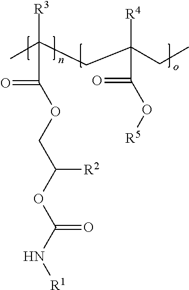

where each R¹ is individually a monovalent organic group, each R² is a individually hydrogen atom or a monovalent organic group, each R³ is individually a hydrogen atom or an alkyl group, each R⁴ is individually a hydrogen atom or an alkyl group, each R⁵ is individually a hydrogen atom or a monovalent organic group, n is from about 5 units to about 500 units, and o is from about 50 units to about 5000 units. In certain embodiments, the alkyl group, R³ or R⁴, is a methyl group. In these or other embodiments, n may be from about 10 units to about 250 units, and in other embodiments from about 50 to about 100 units. In these or other embodiments, o may be from about 100 units to about 2500 units, and in other embodiments from about 500 to about 1000 units.

In one or more embodiments, when a urethane-functional (meth)acrylate monomer, styrene monomer, and a (meth)acrylate or (meth)acrylic acid is used to prepare a polymer, the polymer may be defined by the formula:

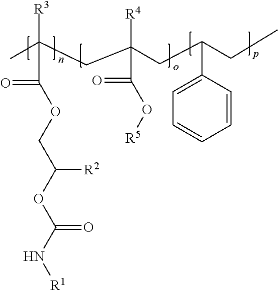

where each R¹ is individually a monovalent organic group, each R² is a individually hydrogen atom or a monovalent organic group, each R³ is individually a hydrogen atom or an alkyl group, each R⁴ is individually a hydrogen atom or an alkyl group, each R⁵ is individually a hydrogen atom or an organic group, n is from about 5 units to about 500 units, o is from about 50 units to about 5000 units, and p is from about 50 units to about 5000 units. In certain embodiments, the alkyl group, R³ or R⁴, is a methyl group. In these or other embodiments, n may be from about 10 units to about 250 units, and in other embodiments from about 50 to about 100 units. In these or other embodiments, o may be from about 100 units to about 2500 units, and in other embodiments from about 500 to about 1000 units. In these or other embodiments, p may be from about 100 units to about 2500 units, and in other embodiments from about 500 to about 1000 units.

In one or more embodiments, when a urethane-functional (meth)acrylate monomer, a methacrylate or methacrylic acid, and an acrylate or acrylic acid is used to prepare a polymer, the polymer may be defined by the formula:

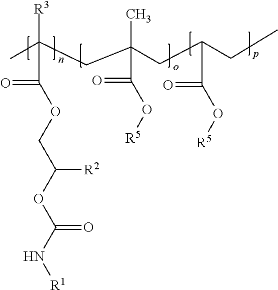

where each R¹ is individually a monovalent organic group, each R² is a individually hydrogen atom or a monovalent organic group, each R³ is individually a hydrogen atom or an alkyl group, each R⁵ is individually a hydrogen atom or a monovalent organic group, n is from about 5 units to about 500 units, o is from about 50 units to about 5000 units, and p is from about 50 units to about 5000 units. In certain embodiments, the alkyl group, R³, is a methyl group. In these or other embodiments, n may be from about 10 units to about 250 units, and in other embodiments from about 50 to about 100 units. In these or other embodiments, o may be from about 100 units to about 2500 units, and in other embodiments from about 500 to about 1000 units. In these or other embodiments, p may be from about 100 units to about 2500 units, and in other embodiments from about 500 to about 1000 units.

As indicated above, urethane-functional (meth)acrylate monomers may be polymerized in an emulsion polymerization. The urethane-functional (meth)acrylate monomers, and optionally co-monomer, may be polymerized through emulsion polymerization to form a latex. In these or other embodiments, an emulsion polymerization system may be prepared by combining a urethane-functional (meth)acrylate monomers, optionally a co-monomer, and a surfactant or a polymerizable surfactant with an initiator or polymerization system in water. The initiator or polymerization system may be introduced to the water along with the urethane-functional (meth)acrylate monomer after the urethane-functional (meth)acrylate monomer is introduced. In one or more embodiments, a seed latex may be prepared or added to the emulsion polymerization mixture prior to or contemporaneous with the addition of the monomer. The emulsion polymerization system should be mixed during the polymerization. In one or more embodiments, the formation of a micelle in the emulsion polymerization system is assisted through sonication.

The emulsion polymerization of a urethane-functional (meth)acrylate monomer may be performed a batch or a semibatch process. Those skilled in the art will recognize that a batch process is a process where all the starting materials are added at the beginning. For this reason, a batch process may be more convenient than a semibatch process. Conversely, a semibatch process is a process where one or more reactants are added over time. A semibatch process, especially under the monomer starved condition, provides may be used to control the kinetics, particle size distribution and morphology during polymerization reactions.

In one or more embodiments, the amount of the surfactant or a polymerizable surfactant may be characterized by the molar percent of the total monomer content (i.e. the moles of surfactant divided by the moles of monomer multiplied by 100). In one or more embodiments, the amount of surfactant may be from about 0.5% to about 5%, in other embodiments from about 1% to about 4%, and in other embodiments from about 2% to about 3%. In other embodiments, the polymerization may take place in the absence of a conventional emulsion polymerization surfactant.

Suitable surfactants include anionic, nonionic, and cationic surfactants. Specific examples of surfactants include, but are not limited to fatty acids, sodium dodecyl sulfate, alkylarylpolyether sulfonates, polyethylene glycol p-(1,1,3,3-tetramethylbutyl)-phenyl ether, and octaethylene glycol monododecyl ethers.

The amount of initiator in may be characterized by molar percent of the total monomer content. In one or more embodiments, the amount of initiator may be from about 0.1% to about 5%, in other embodiments from about 1% to about 4%, and in other embodiments from about 2% to about 3%.

In one or more embodiments, the initiator is a water-soluble free radical initiator. Suitable water-soluble free radical initiators include 4,4′-azobis(4-cyanovaleric acid), potassium persulfate, sodium persulfate, ammonium persulfate, 2,2′-Azobis[2-(2-imidazolin-2-yl)propane], and 2,2′-Azobis(2-methylpropionamidine)dihydrochloride.

The selection of parameters may vary based upon the polymerization system, monomer and optionally co-monomer, and concentrations thereof. Generally, an emulsion polymerization may take place at a temperature of about 30° C. to about 90° C. for about 1 to about 8 hours.

In one or more embodiments, a polymer particle may be prepared by polymerizing a urethane-functional (meth)acrylate monomer via an emulsion polymerization. In these or other embodiments, the polymer of the polymer structure may be described by the polymer structures shown above. In these or other embodiments, the polymer particle may be characterized by the size of the particle. The particle size may be determined by dynamic light scattering were carried out at 25° C. and at a fixed angle of 90° on very diluted emulsions (<0.1 vol %). In one or more embodiments, the polymer particle may be at least 50 nm, in other embodiments at least 100 nm, and in other embodiments at least 150 nm. In one or more embodiments, the polymer particle is at most 1000 nm, in other embodiments at most 800 nm, and in other embodiments at most 700 nm. In one or more embodiments, the polymer particle is from about 50 nm to about 1000 nm, in other embodiments from about 100 nm to about 800 nm, and in other embodiments from about 150 nm to about 700 nm.

In one or more embodiments, the urethane-functional (meth)acrylate monomer may be used to prepare a polymer particle may have one or more layers. A polymer particle with multiple layers may be referred to as a core-shell polymer particle. In these or other embodiments, the core-shell polymer particle may have one or more intermediate layers between the core and the shell. Generally, a core-shell particle may be prepared by charging a first monomer or mixture of monomers and allowing it to polymerize to form a core. Subsequent monomers of mixtures of monomers of a different composition are then added to form additional layers or a shell. Alternatively, in a semi batch process, a core-shell particle may be prepared by gradually adding a first monomer or mixture of monomers and allowing it to polymerize to form a core, and then changing the composition of the monomer added to form additional layers or a shell.

In one or more embodiments, a core-shell polymer particle may include a unit derived from a urethane-functional (meth)acrylate monomer in at least one location. In one or more embodiments, a unit derived from a urethane-functional (meth)acrylate monomer may be in the core of the core-shell polymer particle. In one or more embodiments, a unit derived from a urethane-functional (meth)acrylate monomer may be in the shell of the core-shell polymer particle. In one or more embodiments, the core-shell polymer particle includes one or more intermediate layers and at least one of the intermediate layers includes unit derived from a urethane-functional (meth)acrylate monomer. In one or more embodiments, a unit derived from a urethane-functional (meth)acrylate monomer may be in the core, shell, and any optional intermediate layers of the core-shell polymer particle. The description of the parts per hundred of monomers above may be used to describe individual layers (i.e. core, intermediate layer, or shell) of a core-shell particle.

Those skilled in the art will appreciate that a latex that includes a polymer particle that results from the emulsion polymerization may be used to produce a latex film. Generally, the latex may be cast and then dried at room temperate or using convention drying methods.

While particular embodiments of the invention have been disclosed in detail herein, it should be appreciated that the invention is not limited thereto or thereby inasmuch as variations on the invention herein will be readily appreciated by those of ordinary skill in the art. The scope of the invention shall be appreciated from the claims that follow.

### EXAMPLES

### Materials

Ethylene carbonate (EC, 98%), butyl amine (BA, 99%), methacrylic anhydride (MAA, 94%), dichloromethane (99%), hydroquinone (99%), triethyleneamine (TEA, 99%), dimethyl sulfoxide-d6 (DMSO-d6, 100%), anhydrous magnesium sulphate (99%), hydrochloric acid (HCl, 36.5-38 wt %), methyl methacrylate (MMA, 99%), butyl acrylate (BA, 99%), ammonium persulfate (APS), sodium bicarbonate (NaHCO3), Triton X-200 and Triton X-100, all purchased from Sigma-Aldrich, were used as received. 4-(dimethylamino) pyridine (DMAP, 99%) was obtained from Acros Organics. Acrylic monomers were purified by using inhibitor removal resin (Alfa Aesar) before use. The purified monomers were stored in the refrigerator before synthesis. Deionized water with conductivity below 15 μS/cm was used in the preparation of the latexes.

### Instrumentation

The nuclear magnetic resonance (NMR) spectra were taken in a Varian Mercury 300 MHz spectrometer for liquid samples and Bruker Avance III 300 NMR spectrometer for solid samples. Fourier Transform Infrared Spectroscopy was obtained on a Nicolet 380 FT-IR instrument (Thermo Electron Corp.). Electrospray ionization (ESI) mass spectra were acquired on a HCT Ultra II quadrupole ion trap mass spectrometer (Bruker Daltonics, Billerica, Mass.). Gas Chromatography (Varian CP-3800) was used to detect the unreacted monomers. Particle size and distributions were obtained on dynamic light scattering (DLS) using a PSS NICOMP (Santa Barbara, Calif.) equipped with a He—Ne laser operating at 652 nm and a triple detector. Particle imaging was performed on JSM-1230 TEM (JEOL). PC 700 Benchtop meter (Oakton) was employed for pH and conductivity. Thermal analysis was performed using 1000 DSC (Q1000, TA Instruments). Tensile tests were performed on an Instron 5567 (Instron Corp., Grove City, Pa.). The viscoelastic properties were measured on a dynamic mechanical thermal analyzer (DMTA, Q800, TA Instruments).

### Latex Preparation

### Synthesis of the Non-Isocyanate Urethane Functional Methacylate Monomer (BEM) and its Homopolymer

The synthesis process of BEM consists of two steps (Scheme 1). In a typical procedure to prepare the BEM non-isocyanate urethane methacrylate monomer, first, ethylene carbonate (88.06 g, 1.00 mol) was dissolved in 300 mL dichloromethane in a 1 L three-neck flask. Then butylamine (80.46 g, 1.10 mol) was dropwise added into EC-CH₂Cl₂ solution mixture at 0° C. by using the ice bath under N₂ atmosphere and magnetic stirrer. The mixture was then stirred at room temperature for 24 h. The slightly yellow liquid (yield: 98%), hydroxyalkylcarbamate was obtained after rotary evaporation of the dichloromethane. In the second step, hydroxyalkylcarbamate, BA-EC (80.6 g, 0.5 mol) was dissolved in 300 mL dichloromethane at 0° C. under N₂ atmosphere and magnetic stirrer. 4-(dimethyl-amino) pyridine (DMAP) catalyst (610 mg, 5 mmol), and hydroquinone inhibitor (98.4 mg, 0.8 mmol) were then added, followed by dropwise addition of triethylene diamine (TEA) (70.8 g, 0.7 mol), and then dropwise addition of methacrylic anhydride (98.4 g, 0.6 mol). The reaction mixture was stirred at 0° C. for 24 h. After 24 h reaction, 200 mL dichloromethane was added. The extraction of final products BA-EC-MAA was done with the following steps: the saturated brine (300 mL) was added to get two phase separated mixture; the product (bottom layer) was collected, washed with 1M hydrochloric acid solution (300 ml×3) saturated sodium bicarbonate solution (300 ml×3) and saturated brine (300 mL×1), and dried in anhydrous magnesium sulphate. After the dichloromethane was evaporated, the product was put into the vacuum oven until there is no change of weight. A light yellow liquid product was obtained with yield around 60-70%.

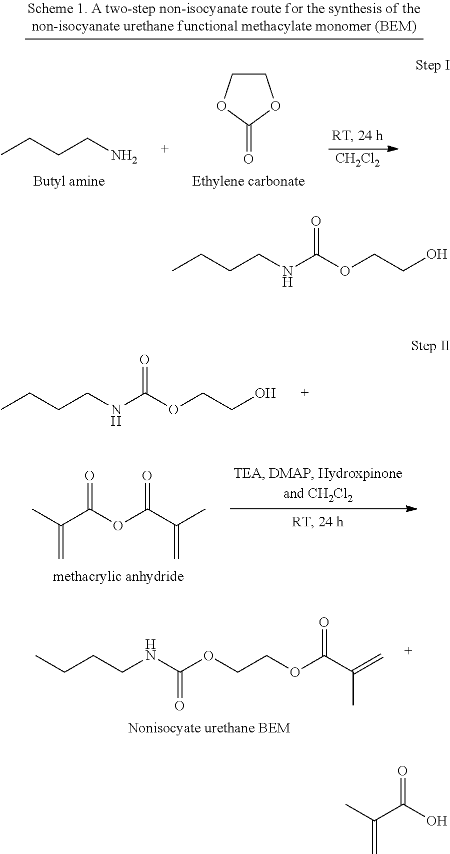

### Synthesis and Design of Latexes

The latexes designed with different polymer compositions, different locations of functionality and different concentration of functionality were synthesized by seeded monomer starved semi-continuous emulsion polymerization with monomer pre-emulsion feed. All seeds used for preparing all the latexes were obtained from single seed latex prepared batch-wise.

Seed: A batch reaction was used to prepare the seed latex. NaHCO₃ (1.5 g) and Triton X-200 (0.5 g) as solution in water (150 g) were added to a 500 mL four-neck flask equipped with a condenser and mechanical stirrer under nitrogen atmosphere. This solution was heated to 75° C.; then a pre-emulsion of monomers was charged to the flask. The pre-emulsion contained BA (41.68 g, 0.33 mol) and MMA (38.32 g, 0.38 mol) with NaHCO₃ (0.1 g) and Triton X-200 (3.6 g). A 2 wt % aqueous solution of ammonium persulfate (102 g) were then charged to the flask. Polymerization was allowed to progress for another 90 min at 75° C.

The same experimental apparatus for seed preparation was used for preparation of the following latexes. The seed (32 g) was charged in the reactor and heated to 75° C. For batch mode latex with 10 wt % urethane functionality (BFL10%), the pre-emulsion and the initiator solution were added once. For semibatch mode latex with 10 wt % urethane functionality (SBFL10%) and the series of latexes with homogeneously distributed urethane functionality among particles (FLH latexes) prepared by semi-continuous process, a pre-emulsion of BA, MMA and BEM (if required) along with an initiator solution along with an initiator solution (2 wt % APS solution, 102 g) were fed continuously for 240 min. Table 1 presents the components and amounts for latex formulations. The mixture was heated at 75° C. and stirred for additional 240 min after the pre-emulsion feed was complete. For core-shell latexes, the core synthesis was prepared the same way as above with monomer composition for core phase (Table 1). Then half of the core product was added to the reactor for preparation of the shell latex. Reactor was heated up to 75° C. Monomer composition for shell phase (Table 1) and the APS solution (2 wt % APS solution, 102 g) were fed during shell synthesis for 240 min. After feeding was over, reaction was run for 240 min to complete conversion.

TABLE 1
Composition of pre-emulsion for urethane-functionalized latexes

| | [BEM] | DI water | NaHCO₃ | Triton | | |
|---|---|---|---|---|---|---|
| | Wt % | (g) | (g) | X200, X100 | Monomers | |
| | | | | | MMA:BA:BEM(g) | |
| BFL10% | 10% | 80 | 0.1 | 3.6, 0.4 | 32:40:8 | |
| SBFL10% | 10% | 80 | 0.1 | 3.6, 0.4 | 32:40:8 | |
| | | | | | Monomer amount | |
| | | | | | MMA:BA:BEM(g) | |
| | | | | | Core | Shell |
| | | | | | Stage | Stage |
| | | | | | (20° C.) | (−20° C.) |
| FLC-S | 0% | 80 | 0.1 | 3.6, 0.4 | 48:32:0 | 25.6:54.4:0 |
| | 5% | 80 | 0.1 | 3.6, 0.4 | 43.6:28.4:8 | 26.4:53.6 |
| | 10% | 80 | 0.1 | 3.6, 0.4 | 37.6:26.4:16 | 26.4:53.6 |
| | 20% | 80 | 0.1 | 3.6, 0.4 | 28.1:19.9:32 | 26.4:53.6 |
| C-FLS | 5% | 80 | 0.1 | 3.6, 0.4 | 48:32 | 22:50:8 |
| | 10% | 80 | 0.1 | 3.6, 0.4 | 48:32 | 16:48:16 |
| | 20% | 80 | 0.1 | 3.6, 0.4 | 48:32 | 6.5:41.5:32 |

| | | | | | |
|---|---|---|---|---|---|
| FLH | 0% | 80 | 0.1 | 3.6, 0.4 | 73.6:86.4:0 (0 mol %) |
| | 5% | 80 | 0.1 | 3.6, 0.4 | 70:82:8 (2.69 mol %) |
| | 10% | 80 | 0.1 | 3.6, 0.4 | 64:80:16 (5.53 mol %) |
| | 20% | 80 | 0.1 | 3.6, 0.4 | 54.5:73.5:32 (11.77 mol %) |

### Characterization of the New Non-Isocyanate Methacrylate Monomer (BEM)

The ¹H, ¹³C NMR spectra were obtained on a Varian Mercury 500 MHz spectrometer using deuterated DMSO as the solvent. Electrospray ionization (ESI) mass spectra were acquired on a HCT Ultra II quadrupole ion trap mass spectrometer with sample concentration of 0.03 mg/ml in CH₂Cl₂:MeOH 50:50 with addition of 1 mg/ml sodium trifluoroacetate (1% volume in sample solution). Fourier Transform Infrared Spectroscopy was obtained on a Nicolet 380 FT-IR instrument using a KBr crystal plate with very thin layer of a liquid sample.

Molecular weight was determined by gel permeation chromatography (GPC) using high-resolution Waters columns with THF at 1 mL/min. The glass transition temperature (Tg) of the final polymer samples was measured by differential scanning calorimetry. Two cycles were performed at cooling and heating rates of 20° C./min. The Tg was obtained from the second cycle.

### Latex Characterization

Latexes were cleaned using a dialysis membrane to remove excess amount of surfactant and other water-soluble ionic materials. A regenerated cellulose dialysis membrane (MWCO 12000-14000) was cleaned to remove soluble residual materials and was rinsed thoroughly with distilled water. A weighted amount of latex was placed inside the membrane and into a container with distilled water. Water was replaced every 12 h until the conductivity of the external water was approximately 0.02 μS.

### Fourier Transform Infrared (FT-IR) Spectroscopy

A thin latex film for FT-IR was directly coated onto the ZnSe plates and dried in the vacuum oven until no weight loss and measured with wavelength ranging from 500 to 4000 cm⁻¹.

### Particle Size Analysis

The measurement of particle sizes and particle size distributions by dynamic light scattering were carried out at 25° C. and at a fixed angle of 90° on very diluted emulsions (<0.1 vol %).

### Conversion Analysis

Total overall and instantaneous monomer conversions were determined gravimetrically from solid content. 1 g of hydroquinone-quenched samples was weighed into aluminum dishes and dry in oven for 2 h at 110° C. The instantaneous conversions of each specific monomer were determined from Gas Chromatography with respective standard calibration curves.

### Morphological Analysis

The morphology of the latex particles was observed on a transmission electron microscope (JEOL 1200EX). The emulsions prepared were diluted with deionized water to about 0.5 wt %. One drop of the diluted emulsion was placed on the coated side of a 400-mesh copper grid and set to dry for 2 h at room temperature. Samples on grids were exposed to RuO₄ vapors for 15 min and dried under ambient conditions for 24 h prior to imaging.

### Film Preparation and Characterization

The latex films were formed by drying the latexes at room temperature. Latexes were cast onto a leveled polytetrafluoroethylene plate and cured at room temperature for 3 days. Films were removed from the plate and kept for additional 7 days at room temperature before testing. Smooth films of constant thickness were obtained. The thickness of the films was around 0.35-0.40 mm. Mechanical properties were tested with 10 specimens (length 40 mm, width 13-15 mm and thickness 0.35-0.40 mm) for each sample at room temperature with a crosshead speed of 10 mm/min applied. An average value of at least ten replicates of each sample was taken. The dynamic mechanical behavior were measured with a frequency of 1 Hz in tensile mode and a heating rate of 3° C./min over a range of −50 to 200° C. The gap distance was set at 2 mm for three rectangular test specimens (length 15 mm, width 10 mm and thickness 0.35-0.40 mm). An average value of at least five replicates of each sample was taken. The thermal stability was characterized with a heating rate of 20° C./min. In order to erase the thermal history effects from the samples, the temperature was equilibrated at 150° C. at the beginning of each experiment. Molecular dynamics was studied by Solid-State NMR (SS-NMR): ¹³C CP MAS experiments were performed on Bruker Avance III 300 NMR spectrometer with a 4 mm double resonance VT CPMAS probe. The ¹H and ¹³C frequencies were 300.1 and 75.5 MHz, respectively. The MAS frequency was set to 12000±5 Hz. The ¹H 90° pulse length was set to 3.75 μs. High-power two pulse phase modulation (TPPM) decoupling with a field strength of 65 kHz was applied to ¹H channel during acquisition. The cross-polarization (CP) contact time and recycle delay were 1.5 ms and 2 s. Each spectrum was obtained by 2048 scans at various temperatures. Lorentz peaks were applied for spectral fitting. The chemical shift was referenced to the CH signal of adamantine (29.46 ppm) as an external reference. The temperature inside of NMR probe was carefully calibrated using the temperature dependence of the ²⁰⁷Pb chemical shift of Pb(NO₃)₂.

### Homopolymer of Urethane Acrylate Monomers (DSC and GPC)

A series of homopolymer of BEM (PBEM) with low to high molecular weights were synthesized with solution polymerization with AIBN as the initiator and as the chain transfer agent. The glass transition temperature of PBEMs was determined by DSC. The glass transition temperature, which is independent of molecular weight above a specific molecular weight, is defined as the glass transition temperature of a homopolymer. The glass transition temperature was dependent of number average molecular weight before the number average molecular weight reached 100,000 g/mol. Then there was a glass transition temperature plateau after 100,000 g/mol. The glass transition temperature at the plateau as defined, which was around 26° C., was the glass transition temperature of BEM's homopolymer, PBEM.

### The Composition of Urethane Functional Latex

### Kinetic Analysis During Polymerization

Two latexes poly(MMA/BA/BEM) with the same monomer composition (MMA/BA/BEM: 32/40/8) were prepared with different techniques. The first one was obtained by using the semibatch emulsion polymerization while the second one was prepared through simple batch emulsion polymerization. Overall conversion and particle size increased during synthesis for both BFL 10% and SBFL10% latexes. In the initial stage of batch polymerization, there was an instantaneous increase of the particle number due to the presence of high concentration of monomer and initiator. The number of particles (Np) from batch polymerization was higher than the results obtained from the semi-batch polymerization. In other words, the particle size from the semi-batch polymerization was also higher than the results from batch polymerization. For semi-batch polymerization, the instantaneous overall conversion was above 85% after initial several minutes, which indicated that the monomer-starved condition had been reached. The rate of polymerization under monomer-starved condition was relatively stable before it dropped down while the rate of polymerization from batch polymerization increased very quickly at the initial stage and then decreased slowly.

The instantaneous conversion (Xinst.) of each monomer was calculated based on the residue monomers detected by GC. In the semibatch emulsion polymerization, the monomers, MMA, BA and BEM showed comparable instantaneous conversion. At the very beginning, the initial instantaneous conversions of MMA, BA and BEM were around or above 85%, which indicated that the monomer-starved condition had been reached. The polymer chains, therefore, were homogeneously composed of MMA, BA and BEM based on the composition of feeding monomers. In the batch emulsion polymerization, there is significant difference between the instantaneous conversions of MMA, BA and BEM. The initial instantaneous conversions of MMA and BEM were around 80%. The monomer, BA showed lower instantaneous conversions than that of MMA and BEM. In comparison, it took only several minutes for instantaneous conversions of MMA and BEM to reach 80% while almost 20 minutes for instantaneous conversion of BA. Therefore, BA showed a slower reactivity in its copolymerization with MMA and BEM in the batch polymerization. The difference in the instantaneous conversions of MMA, BA and BEM indicated the existence of two rich phases: one is the rich phase of MMA and BEM and the other one is rich phase of BA.

### Thermal Properties

The thermal property of the latexes was studied by DSC. The glass transition temperatures of two latexes prepared from semibatch and batch polymerization, and the results are listed in Table 2. The theoretical glass transition temperature was calculated based on the Fox equation with Tg, MMA=105° C., Tg, BA=−54° C. and Tg, BEM=26.5° C. The theoretical glass transition temperature was designed around 0° C. There was a little deviation for the experimental glass transition temperatures of both latexes from the theoretical values, i.e. 4.3° C. for BFL10 wt % and 4.6° C. for SBFL10 wt %. However, the experimental glass transition temperatures of BFL10 wt % and SBFL10 wt % were very close due to the same composition of monomer feeding. There was only one glass transition showed from the heat flow curves for both of the latexes. However, from the heat flow curves with the same heating rate, it was observed that the glass transition of the latex from batch polymerization was broader than the result of the latex from semibatch polymerization. In comparison, the sharp glass transition can represent a relatively homogeneous SBFL10 wt % while the broad one for the BFL10 wt %.

TABLE 2
Overall result of glass transition temperatures of BFL10% and SBFL10%

| | | Experimental Tg | Experimental Tg |
|---|---|---|---|
| | Theoretical Tg | from DSC | from DMA |
| BFL10% | 0° C. | 4.3 ± 1.0° C. | 40 ± 1.0° C. |
| SBFL10% | 0° C. | 4.6 ± 1.0° C. | 34 ± 1.0° C. |

### Mechanical Properties

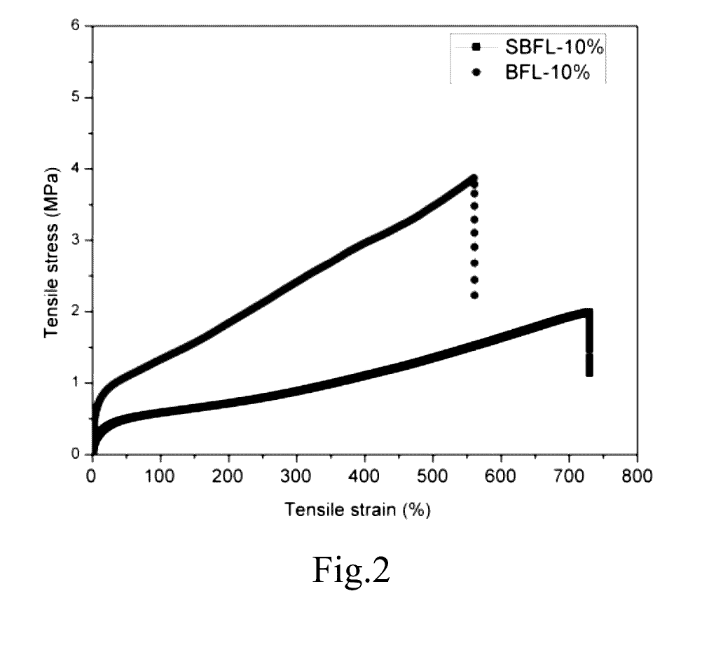

The mechanical properties of two latexes were evaluated by tensile tests. The tensile properties of latexes prepared from semibatch and batch polymerization were compared in FIG. 2. The latex film prepared from BFL10% showed larger modulus and tensile strength while smaller elongation-at-break than that from SBFL10%. From the stress-strain curves, the terpolymer from batch polymerization had an initial rapid build in stress at small elongations. Then the terpolymer film elongated further to the end of the breakage of the film. On the other hand, the terpolymer from semibatch polymerization exhibited a gradual increase of the stress and strain to the end of the breakage of the film. The modulus and tensile strength for the latex from batch polymerization were 3.7 and 4.2 MPa, respectively, which were 30% and 130% higher than that from the semibatch polymerization with 3.0 and 1.8 MPa, respectively. However, the 570% elongation-at-break observed from batch polymerization was lower than 720% from semibatch polymerization.

### Viscoelastic Properties

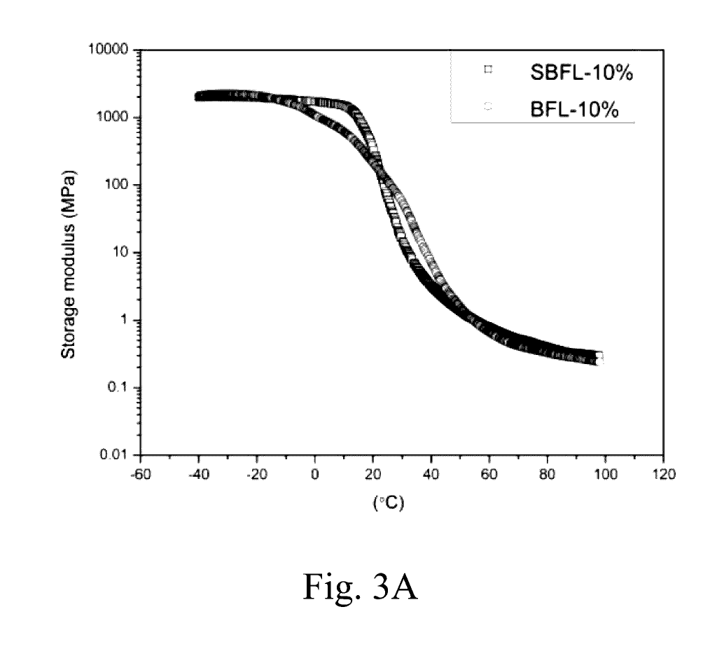

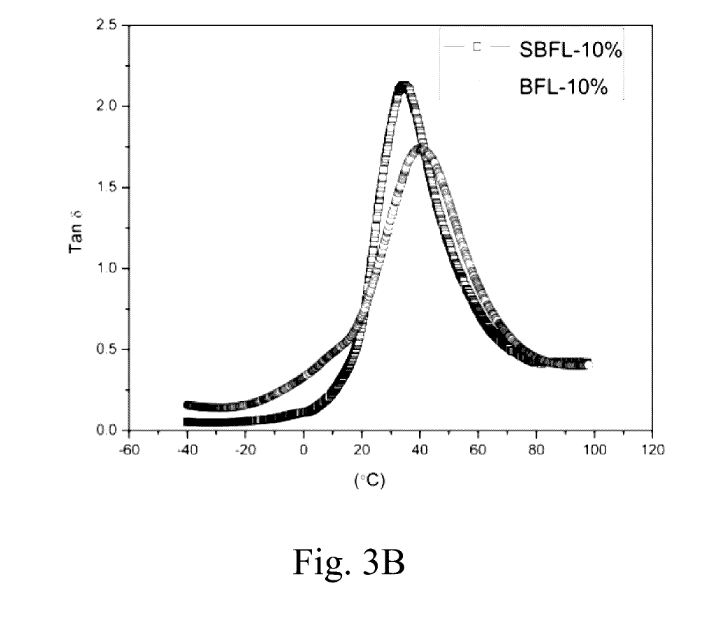

The dynamic properties were studied by DMA. The viscoelastic properties of latexes prepared from semibatch and batch polymerization were compared in FIGS. 3A, 3B, and 3C. The terpolymer from batch polymerization showed a higher storage modulus in the glassy state than that from semibatch polymerization. The storage modulus of the latex film prepared from batch polymerization in the glassy state was 2102 MPa while that from semibatch polymerization was 1969 MPa. In addition, it was observed from the E′ curves that the glass transition was broader for the latex from semibatch polymerization than that from batch polymerization. In the tan δ curves, the glass transition temperatures were obtained from the position where the maximum tan δ located. The glass transition temperatures were 40° C. and 34° C. for latexes from batch and semibatch polymerization, respectively. In addition, the comparison of the full-width-at-half-maximum tan δ values obtained from the tan δ curves indicated significant difference between batch and semibatch polymerization. The full-width-at-half-maximum tan δ was 40° C. for batch polymerization, while only 26° C. for semibatch polymerization. The broader tan δ curve and E′ curve of BFL10% than that of SBFL10% represented a more homogeneous composition.

### The Urethane Functional Latexes with Homogeneous and Core-Shell Structures

### Composition of Urethane Functionality

The composition of urethane latexes were studied by qualitatively analysis of FTIR spectra. The vibrations at 1720 cm-1 was overall sum of the stretching of carbonyl groups (C═O) from each monomer. The vibrations at 1530 cm-1 and at 3350 cm-1 were characteristics of the stretching and bending of urethane groups (NH) only from urethane methacrylates. In the qualitatively analysis, the stretching of carbonyl groups C═O at 1720 cm-1 were referenced as the internal standard while the stretching of urethane (NH) at 3350 cm-1 and the bending of urethane (NH) at 1530 cm-1 were as the characteristic of urethane functionality from urethane methacrylates. The theoretical ratios of N—H to C═O are 0, 2.62, 5.24 and 10.53 mol %, respectively. The spectra showed the qualitative increase of the urethane functionality of latexes from low concentration to high concentration in the FLH series, FLC-S series and C-FLS series.

### Particle Size and Morphology

The particle size was measured by DLS and TEM and compared with the theoretical result to evaluate the stability of the dispersion. The overall results of particle size were listed in Table 3. The theoretical particle size was around 380 nm, which was calculated based on the mass balance with the equation below.

d_{f}=(1+Wₘ/Wₛ)^{1/3}dₛ

Where Wm is the amount (g) of monomer feed; Ws is the amount (g) of seed particles; ds and df is the particle diameter (nm) of the seed and final latexes, respectively.

DLS and TEM were combined to use in this study to check the particle size and its distribution. As the most widely used method, DLS directly gives the z-average (dz) particle sizes. To compare with theoretical results, dz was listed in the Table 3. The particle size dv from DLS showed a little difference compared with the theoretical values. In addition, the particle sizes for all latexes were comparable within the error. The particle size was further checked with TEM. The contrast of dark and white in TEM images showed the morphology of the core-shell structures. The particle size from TEM was much larger than DLS and theoretical results. This was not consistent with other studies of TEM particle sizes. The inconsistency probably resulted from the flattening due to the low Tg.

TABLE 3
Particle size results from DLS and theoretical calculation

| | |
|---|---|
| DLS | Theoretical |

| | d_{z}/nm | PDI | dᵥ/nm |
|---|---|---|---|
| PA(FLH0%) | 377 | 0.01 | 380 |
| C-S 0% | 380 | 0.01 | 380 |
| FLH5% | 394 | 0.01 | 380 |
| FLH10% | 391 | 0.02 | 380 |
| FLH20% | 390 | 0.03 | 380 |
| FLC-S5% | 384 | 0.02 | 380 |
| FLC-S10% | 383 | 0.03 | 380 |
| FLC-S20% | 374 | 0.03 | 380 |
| C-FLS5% | 385 | 0.02 | 380 |
| C-FLS10% | 380 | 0.01 | 380 |
| C-FLS20% | 390 | 0.01 | 380 |
| Uncertainty | ±5 | ±0.01 | — |

### Thermal Properties

The thermal property of the latexes was studied by DSC. The glass transition temperatures of a series of latexes prepared from semibatch polymerization, and the overall results are listed in Table 4. The theoretical glass transition temperature was calculated based on fox equation with Tg, MMA=105° C., Tg, BA=−54° C. and Tg, BEM=26.5° C. The core-shell latex was designed with Tg, overall=0° C., Tg, core=20° C. and Tg, shell=−20° C. The latexes with homogeneous structure were designed with Tg=0° C., the same as the overall Tg of core-shell latexes. The morphology of core-shell and homogeneous structure were indirectly proved from the glass transitions. From the heat flow curves, it was observed that there was only one glass transition for the latexes with homogeneous structures while two clear glass transitions were observed for the latexes with core-shell structures. There was a little deviation for the experimental glass transition temperatures from the theoretical values, which shifted to high temperatures.

TABLE 4
Overall result of glass transition temperatures of core-shell and
homogeneous latexes

| | T_{g} from DSC | | T_{g} from DMA | |
|---|---|---|---|---|
| Theoretical | T_{g} = 0° C. | | | |
| FLH5% | −1 | | 31 | |
| FLH10% | 2 | | 32 | |
| FLH20% | 4 | | 38 | |
| | Tg, core = | Tg, | Tg, core = | Tg, |
| Core-Shell | 20° C. | shell = −20° C. | 20° C. | shell = −20° C. |
| FLC-S5% | 21 | −12 | 45 | 8 |
| FLC-S10% | 21 | −7 | 51 | 10 |
| FLC-S20% | 26 | −7 | 52 | 11 |
| C-FLS5% | 24 | −14 | 50 | 9 |
| C-FLS10% | 24 | −12 | 51 | 10 |
| C-FLS20% | 30 | −12 | 54 | 13 |
| Uncertainty | ±1 | ±1 | ±1 | ±1 |

### Mechanical Properties

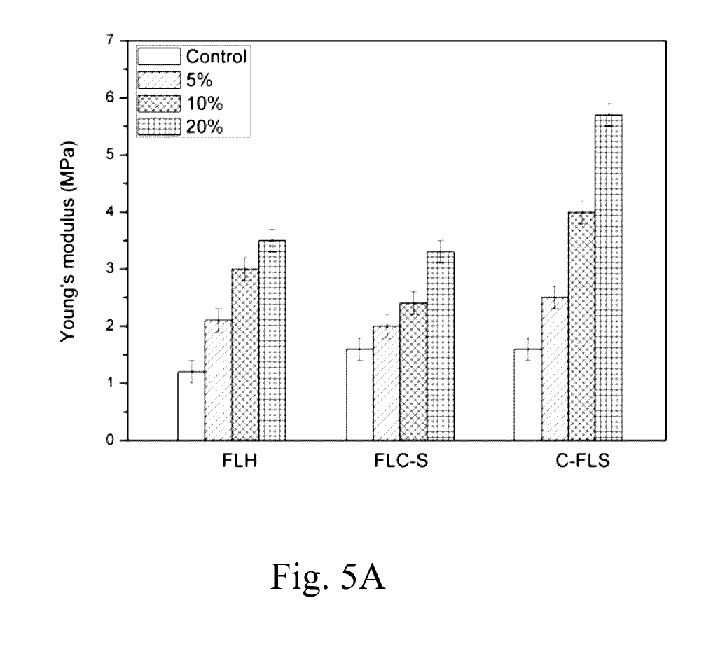

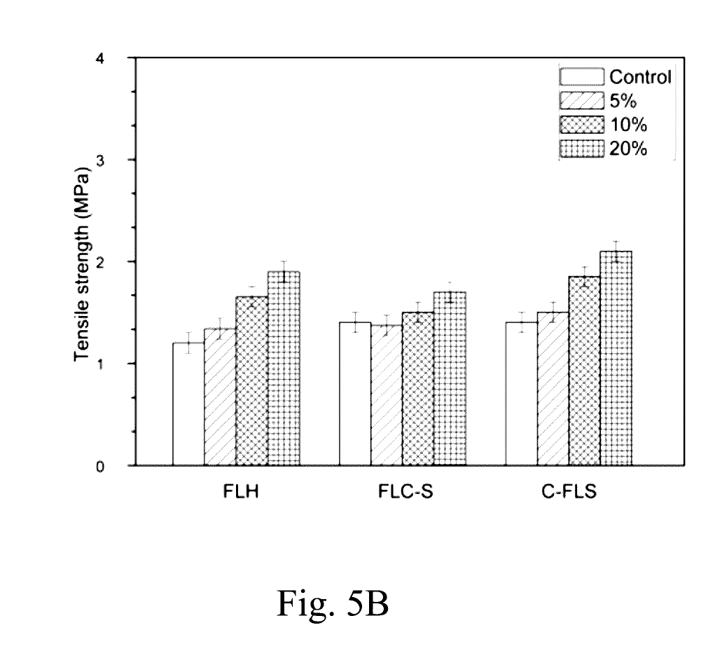

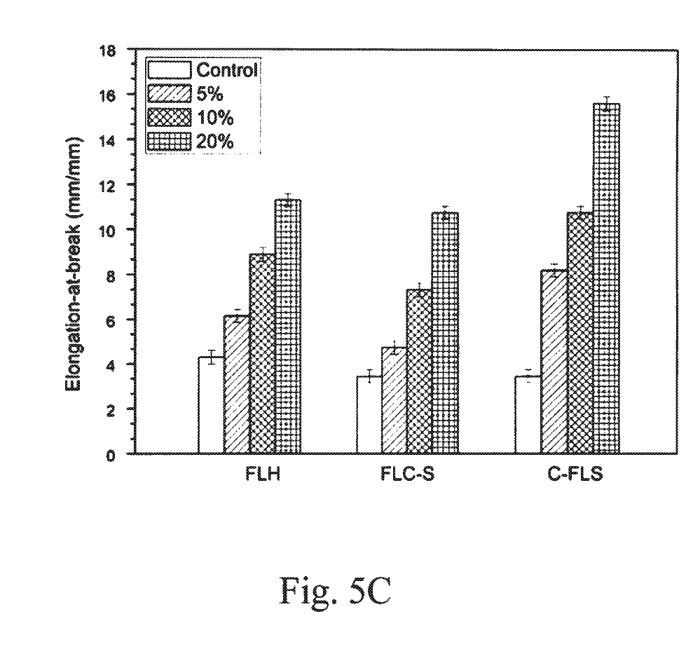

The mechanical properties were evaluated by tensile tests. The tensile properties, modulus, tensile strength and elongation-at-break were compared in FIGS. 5 A, B, and C. among three series of latexes prepared from semibatch polymerization, i.e. FLH, FLC-S and C-FLS.

The tensile strength increased with the addition of urethane functionality. When the urethane functionality was used through the whole particle (FLH), the tensile strength showed a gradually increase with 5 wt % and 10 wt % urethane functionality content, to maximum of 1.9 MPa with 20 wt % urethane functionality content. When the urethane functionality was used in the core stage of the particle (FLC-S), the tensile strength showed no change within 0-5 wt % urethane functionality content and gradually increased within 10-20 wt % urethane functionality content. When the urethane functionality was used in the shell stage of the particle (C-FLS), it showed the similar trend as FLH. The tensile strength showed increase with the amount of urethane functionality content, and reached the maximum at 20 wt % urethane functionality content. When urethane functionality was used in FLH or C-FLS systems, the tensile strength showed a continuous increase with the amount of urethane functionality while initial decrease of tensile strength was found for FLC-S system. The tensile strength of all three systems reached the maximum at 20 wt % reactive diluents content. The maximum tensile strength of FLH system was 1.9 MPa, which was 1.6 times higher than the control sample; and the maximum tensile strength of C-FLS and FLC-S systems were 2.2 MPa and 1.7 MPa, which were 1.6 times and 1.2 times higher than the control sample. In comparison, the tensile strength of FLH system was a little higher than that of the FLC-S system at the same percent loading in the range of 5-20 wt % while the tensile strength of C-FLS system was always 1.1-1.3 higher than that of the FLH or FLC-S system at the same percent loading in the range of 5-20 wt %.

The elongation-at-break showed a continuous increase with the addition of urethane functionality and reached the minimum at 20 wt % urethane functionality content for all three systems. The maximum elongation-at-break of FLH system was 2.6 times higher than the control sample; and the maximum elongation-at-break of C-FLS and FLC-S systems were 3.0 times and 4.5 times higher than the control sample, respectively. In comparison, the elongation-at-break of FLH system was a little higher than that of the FLC-S system at the same percent loading in the range of 5-20 wt % while the elongation-at-break of C-FLS system was always much higher than that of the FLH or FLC-S system at the same percent loading in the range of 5-20 wt %. In the range of 5-20 wt %, the elongation-at-break of C-FLS system was around 1.2-1.4 times and 1.5-1.7 times higher than that of the FLH and FLC-S systems.

The tensile modulus showed a continuous increase with the addition of urethane functionality in the percent loading range of 5-20 wt % and reached the minimum at 20 wt % urethane functionality content for all three systems. The maximum tensile modulus of FLH system was 2.9 times higher than the control sample; and the maximum elongation-at-break of C-FLS and FLC-S systems were 2.0 times and 3.6 times higher than the control sample, respectively. In comparison, the trend of tensile modulus was consistent with the elongation-at-break. The tensile modulus of FLH system was a little higher than that of the FLC-S system at the same percent loading in the range of 5-20 wt % while the tensile modulus of C-FLS system was always much higher than that of the FLH or FLC-S system at the same percent loading in the range of 5-20 wt %. At 5-10 wt %, the tensile modulus of C-FLS system was around 1.2-1.3 times higher than that of the FLH system while at 20 wt %, 1.6 times. At 5 wt %, the tensile modulus of C-FLS system was around 1.3 times higher than that of the FLC-S system while at 10-20 wt %, 1.7 times.

### Viscoelastic Properties

The dynamic properties were studied by DMA. The storage modulus E′ in the glassy state of the latex films increased with addition of the urethane content for all latex systems in the load range of 5-20 wt %. In comparison, the storage modulus of FLC-S, FLH and C-FLS systems showed ascending trend at the same percent loading in the range of 5-20 wt %. From the E′ curves, it was observed that there was only one glass transition for the latexes with homogeneous structures while two clear glass transitions were observed for the latexes with core-shell structures. The same phenomena were observed from the tan δ curves with obvious α-transitions. In addition, the glass transition temperatures can be obtained from the position where the maximum tan δ located from the tan δ curves. One Tg was determined for FLH series of latexes while two Tgs for FLC-S and C-FLS series of latexes from tan δ curves. The results were listed in Table 2. The Tg increased with the addition of urethane functionality content in general. The maximum of α-transition decreased with addition of urethane functionality content as well.

What is claimed is:

1. A monomer defined by the formula 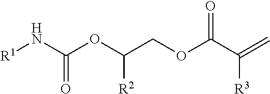
where R¹ is a monovalent organic group, R² is a hydrogen atom or a monovalent organic group, and R³ is a hydrogen atom or an alkyl group.

2. The monomer of claim 1, where the monovalent organic group, R¹, is a hydrocarbon group with 1 carbon atom to about 12 carbon atoms.

3. The monomer of claim 1, where the monovalent organic group, R², is a hydrocarbon group with 1 carbon atom to about 12 carbon atoms.

4. The monomer of claim 1, where R¹ is a hydrocarbon group selected from methyl, ethyl, propyl, isopropyl, isobutyl, tert-butyl, n-butyl, sec-butyl, isopentyl, tertpentyl, n-pentyl, sec-pentyl, terthexyl, n-hexyl, isohexyl, and sec-hexyl, n-heptyl, n-octyl, n-nonyl, n-decyl and n-dodecyl.

5. The monomer of claim 1, where R³ is a hydrogen atom.

6. The monomer of claim 1, where R³ is a methyl group.

7. The monomer of claim 1, where monomer is selected from:

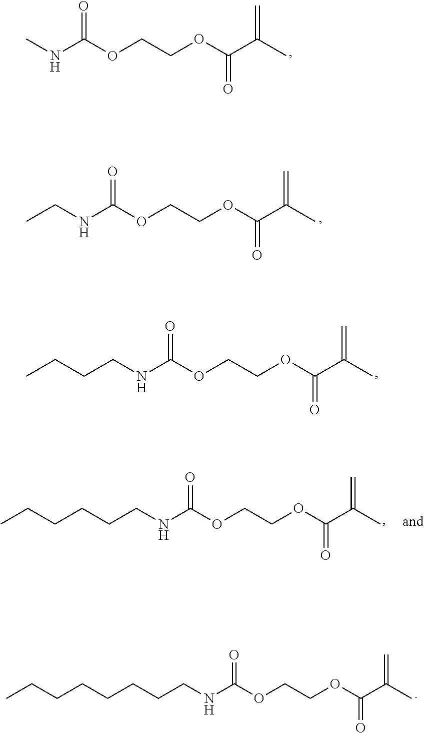

8. A method of preparing a polymer comprising:
polymerizing a urethane-functional acrylate monomer defined by the formula 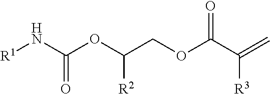
where R¹ is a monovalent organic group, where R² is a hydrogen atom or a monovalent organic group, and R³ is a hydrogen atom or an alkyl group.

9. The method of claim 8, where the polymerization is initiated with a radical initiator.

10. The method of claim 8, where the polymerization is a controlled living polymerization.

11. The method of claim 10, where the controlled living polymerization is selected from atom transfer radical polymerization, reverse atom transfer radical polymerization, reversible addition-fragmentation chain-transfer polymerization, and nitroxide mediated polymerization.

12. The method of claim 8, where the polymerization is performed as an emulsion polymerization.

13. The method of claim 12, where the emulsion polymerization produces a core-shell polymer particle.

14. The method of claim 8, where the polymerization includes one or more co-monomers.

15. The method of claim 14, where the co-monomers are selected from (meth)acrylic acids, (meth)acrylates, and vinyl aromatic compounds.

16. A polymer with a unit defined by the formula: 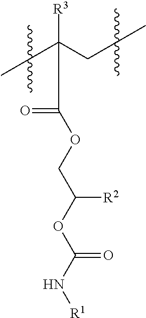
where R¹ is a monovalent organic group, R² is a hydrogen atom or a monovalent organic group, and R³ is a hydrogen atom or an alkyl group.

17. The polymer of claim 16, where the polymer is defined by the formula 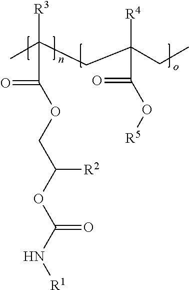
where each R¹ is individually a monovalent organic group, each R² is a individually hydrogen atom or a monovalent organic group, each R³ is individually a hydrogen atom or an alkyl group, each R⁴ is individually a hydrogen atom or an alkyl group, each R⁵ is individually a hydrogen atom or an organic group, n is from about 5 units to about 500 units, and o is from about 50 units to about 5000 units.

18. The polymer of claim 16, where the polymer is defined by the formula 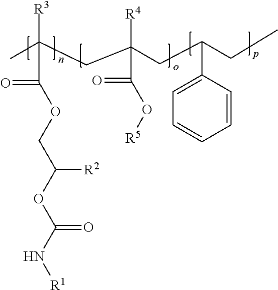
where each R¹ is individually a monovalent organic group, each R² is a individually hydrogen atom or a monovalent organic group, each R³ is individually a hydrogen atom or an alkyl group, each R⁴ is individually a hydrogen atom or an alkyl group, each R⁵ is individually a hydrogen atom or an organic group, n is from about 5 units to about 500 units, o is from about 50 units to about 5000 units, and p is from about 50 units to about 5000 units.

19. The polymer of claim 16, where the polymer is defined by the formula 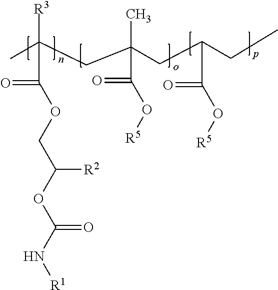
where each R¹ is individually a monovalent organic group, each R² is a individually hydrogen atom or a monovalent organic group, each R³ is individually a hydrogen atom or an alkyl group, each R⁵ is individually a hydrogen atom or a monovalent organic group, n is from about 5 units to about 500 units, o is from about 50 units to about 5000 units, and p is from about 50 units to about 5000 units.

20. A latex comprising a polymer particle with a unit defined by the formula: 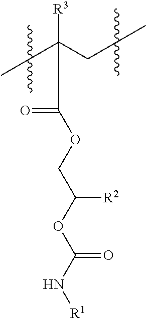
where R¹ is a monovalent organic group, R² is a hydrogen atom or a monovalent organic group, and R³ is a hydrogen atom or an alkyl group.
